(19) United States
(12) Patent Application Publication
(10) Pub. No.: US 2010/0081997 A1
(43) Pub. Date: Apr. 1, 2010

(54) DOSAGE DELIVERY DEVICE

(76) Inventor: Paul Harry Moed, St. Peter Port Guernsey (GB)

Correspondence Address: EDWIN D. SCHINDLER, FIVE HIRSCH AVENUE, P.O. BOX 966, CORAM, NY, 11727-0966, US

(21) Appl. No.: 12/517,768

(22) PCT Filed: Dec. 6, 2007
(86) PCT No.: PCT/GB07/04688

§ 371 Date: Jun. 4, 2009

(30) Foreign Application Priority Data
Dec. 7, 2006 (GB) 0624470.1

(51) Int. Cl. A61M 5/315; A61M 5/31

(52) U.S. Cl. 604/191; 604/228; 604/200; 604/506

(57) ABSTRACT

A dosage delivery device having a housing within which is formed a reservoir, with a plunger located within the reservoir and movable to dispense a material from a discharge opening of the reservoir. The device includes a plunger actuation mechanism, which is movable between a contracted storage position, in which the mechanism is held, at least partially, within the housing and an extended primed position, in which the plunger can be actuated. The dosage delivery device additionally includes a priming mechanism slidably mounted with respect to the housing and movable between a storage and primed position to expose the discharge opening and release the plunger actuation mechanism.

This invention relates to dosage delivery devices and particularly but not exclusively, to such devices for use in the oral, rectal and injection delivery of medicaments. Such devices are of particular value in emergency situations where a dosage of medicament has to be delivered urgently and with minimal preparation of the device prior to use. The device according to the present invention may deliver medicaments, for example, to humans or animals.

Various modern medicines are adapted for oral or rectal delivery, and in prescribed quantities. They can be provided in capsule form, sometimes with the capsule material being soluble so that it dissolves in the user's mouth or rectum. Dosages can also be delivered by aerosol. Some medicines are adapted for delivery via a needle. Dosages in liquid form particularly can be delivered by means of a pump or syringe. Devices of this latter type would include a reservoir with a discharge opening at one end, with a plunger in the reservoir being movable towards the opening to discharge liquid in the reservoir through the opening. The present invention is directed at devices of this type.

In order to move the plunger in the reservoir of a delivery device of the kind just referred to, an actuating member has to extend beyond the reservoir by at least the full stroke of the plunger prior to delivery commencing. If the device is pre-charged with a medicament or other treatment liquid, this means that the actuating member has to project from the reservoir from the time of pre-charging until the time of use. If the device is to be carried by the user for example for use only when an emergency arises, the projecting member generates a potential for damage to the device. Of course, if the device is for emergency use, then any risk of damage to the device has to be avoided as has any risk of accidental discharge of the treatment liquid.

International Patent Application PCT/US02/05114 is directed at an operating mechanism for the plunger in a dosage delivery device of the kind referred to above. The mechanism comprises an actuator slideably mounted with respect to a drive member for driving a plunger within a reservoir. When the device is to be used a dose is set by pulling the actuator along the drive member away from the plunger so as to enable operation of the plunger. The actuator is then pushed and engages the drive member so as to drive the plunger into the reservoir so as to dispense a medicament held therein.

An emergency dosage delivery device of the type described above is generally distributed in a pre-packaged form, which is carried around in case of an emergency. Accordingly, it is important that the device in its packaging is robust, compact and easy to remove from its packaging to a configuration in which a medicament it contains can be dispensed. Often, when a device of the invention has to be used, a covering or closure member has first to be removed to expose the discharge opening through which the medicament is to be dispensed. This can be difficult in an emergency situation. In addition the covering or closure may form a choking hazard.

According to the present invention there is provided a dosage delivery device having a housing defining a reservoir and a plunger within the reservoir moveable to dispense a material from a discharge opening of the reservoir, comprising: a plunger actuation mechanism moveable between a contracted storage position in which the mechanism is held at least partially within the housing and an extended primed position in which the plunger can be actuated; and a priming mechanism slideably mounted with respect to the housing, for example, the priming mechanism may be slideably mounted on the housing, and moveable between a storage and a primed position to expose the discharge opening and to release the plunger actuation mechanism.

The present invention enables priming of a dosage delivery device easily and quickly by movement of a priming mechanism on the housing. The priming action acts to release the plunger actuating mechanism into a position from which the plunger can be actuated and acts to expose a discharge opening in the housing through which a material, such as a medicament, can be directly dispensed, for example orally, rectally or via an injection needle, into a patient. In the storage position, the plunger actuation mechanism is held at least partially within the housing. This helps to improve the compactness of the device in the storage position. Ideally, for improved compactness, the plunger actuation mechanism is held completely within the housing in the storage position.

The plunger actuation mechanism may be biased into the extended position, so that it assumes the extended position on release. This further enhances the efficiency with which the device can be primed.

The plunger actuation mechanism may be held at least partially within the housing by a latch arrangement and the priming mechanism may comprise an abutment surface, for engaging the latch arrangement so as to release the plunger actuation mechanism. The latch arrangement may conveniently be formed as an outwardly facing latch arrangement for engaging a portion of the housing, in which case the abutment surface may comprise an inwardly facing latch arrangement. In addition, the priming mechanism may comprise a latch arrangement engageable with the housing in the primed position. For example, the inwardly facing latch arrangement provided for engaging the outwardly facing latch arrangement of the plunger actuation mechanism may also be engageable with the housing in the primed position. In this way, the inwardly facing latch arrangement of the priming mechanism has a dual function, which provides a simplified design with enhanced functionality.

The housing may have a latch hole arrangement formed within it arranged to engage the latch arrangement on the plunger actuation mechanism in the storage position and to engage the latch arrangement on the priming mechanism in the primed position. Similarly, the latch hole arrangement has a dual function, leading again to a simplified design. In particular, the latch arrangement on the plunger actuation mechanism may engage the latch hole arrangement from one side and the latch arrangement on the priming mechanism may engage the latch hole arrangement from the opposite side to the said one side. Where the housing is generally cylindrical and transitions into an enlarged rearward portion via a stepped portion comprising an outwardly extending step face, the latch holes of the latch hole arrangement may be formed through the step face. The outwardly extending step face enables an approach to the latch hole arrangement from opposing sides of the step face, which is convenient for latching of components, which are slideably mounted with respect to the housing.

The plunger actuation mechanism may comprise an injection sleeve slideably mounted within the housing behind and operably engaged with the plunger and a plunger actuator slideably mounted within the injection sleeve and which is engageable with the housing in the storage position and released from the housing and engageable with the injection sleeve in the primed position so as to actuate the plunger. In this case a spring arrangement may be located within the injection sleeve between the plunger and the plunger actuator for urging the plunger actuator to the primed position when the plunger actuator is released from the housing. This automatically moves the plunger actuator into a primed position in which it can be used to actuate the plunger via the injection sleeve, when the plunger actuator is released.

In the storage position a closure member may cover the discharge opening, in which case a tie may be fixed between the discharge opening and the priming mechanism to facilitate removal of the closure member to expose the discharge opening when the priming mechanism is moved between the storage and primed positions.

The priming mechanism may comprise an operating sleeve slideably mounted on the housing which has a forward end operatively connected to a closure member of the discharge opening and has a rearward end engageable with the plunger actuation mechanism so as to release the plunger actuation mechanism and wherein the priming device may be moved rearwardly along the housing between the storage and the primed positions. In this case, any abutment surface of the operating sleeve may be located at the rear of the operating sleeve.

To improve the safety of the device, the closure member may be stored in a chamber formed between the housing and the priming mechanism in the primed position. This removes the closure member from the vicinity of the device as the closure member may, for example, be a choking hazard. For example the chamber may be formed by a slot in the operating sleeve and the operating sleeve may be operatively connected to the closure member by a tie which extends along the slot from the closure member to a fixing point on the operating sleeve. Then when the operating sleeve is moved from the storage to the primed position, the closure member is removed to expose the discharge opening via the tie, due to the movement of the operating sleeve and then the tie pulls the closure member into the slot for safe storage.

The tie and the closure member may be integrally formed with a forward end of the housing, for example by injection moulding. In this case, the closure member may be connected at one end to the tie and may be connected to the housing via frangible tear lines. For example, the closure member may be permanently connected at an end remote from the tie to the housing via a hinge portion. The design of the housing with the integrally formed tie and closure member reduces the complexity with which the device is assembled due to a reduction in component parts.

The housing may be formed with an enlarged portion at its rearward end and the priming mechanism may be formed with a similarly enlarged portion and a compressible protective sleeve may be mounted between the two enlarged portions, wherein movement of the priming mechanism between the storage and primed positions moves the enlarged portions together compressing the protective sleeve.

Where the dosage delivery device according to the present invention is to be used for the oral delivery of a medicament, the forward end of the housing may be suitably shaped to easily fit within a patient's mouth.

Where the dosage delivery device according to the present invention is to be used for the rectal delivery of a medicament, the housing may be formed at its forward end with a tapering nozzle suitably shaped for rectal insertion and the discharge opening may then be formed at the forward end of the nozzle.

Where the dosage delivery device according to the present invention is to be used for injection delivery of a medicament, the housing may be formed at its forward end with an interface for receiving a luer slip needle attachment or a luer lock needle attachment over the discharge opening.

According to a second aspect of the present invention there is provided a method of operation of the dosage delivery device described above, comprising the steps of: moving the priming mechanism from the stored to the primed position; and actuating the plunger using the plunger actuating mechanism so as to dispense a material from the discharge opening. Where the medicament is to be delivered by needle injection a luer needle attachment may be fitted over the discharge opening of the housing, after the priming step and before the actuating step so as to dispense a material from the luer needle attachment via the discharge opening.

The latch arrangements may be resilient and this resilience is generally provided by forming the priming mechanism and the plunger actuation mechanism, or parts thereof, on which the latches are formed from a suitable resilient material. Suitable resilient materials may include materials suitable to be moulded, for example injection moulded, or otherwise formed into the desired shape, such as plastics, for example thermoplastics.

The device may additionally comprise a secondary reservoir containing the material and which is slideably located within the housing; and an injection needle, which communicates with material within the secondary reservoir and extends forwardly of the secondary reservoir within the housing and which is aligned with the discharge opening of the housing; wherein the plunger is first engageable with the secondary reservoir so as to slide the secondary reservoir forward within the housing and move the injection needle through the discharge opening and is then slideable within the secondary reservoir so as to dispense the material through the injection needle.

The material may be contained within a sealed capsule located within the reservoir. Where the material is a medicament, the sealed capsule maintains a sterile environment for the medicament until it is dispensed. The capsule may have a pierceable forward face and the secondary reservoir may comprise a cutting element for piercing the forward face of the capsule so as to dispense material from within the capsule via the pierced forward face of the capsule to the discharge opening. The cutting element may be located adjacent the discharge opening. This provides a means of dispensing the medicament from the capsule and out of the discharge opening.

For example, the device may additionally comprise a secondary reservoir containing the capsule and which is slideably located within the housing; and an injection needle, having a rearward end forming the cutting element and a forward end which extends forwardly of the secondary reservoir within the housing and which is aligned with the discharge opening of the housing; wherein the plunger may be first engageable within the secondary reservoir so as to slide the secondary reservoir forward within the housing and move the injection needle through the discharge opening, may be secondly engageable with the capsule so as to slide the capsule forward within the secondary reservoir until the rearward end of the needle engages the pierceable forward face of the capsule so as to pierce said forward face and may be thirdly slideable within the capsule so as to dispense the material through the injection needle.

The capsule may be compressed by engagement with the plunger to dispense the material. In this case the plunger may be engageable with the capsule so as to slide the capsule forward within the secondary reservoir until the cutting element engages the pierceable forward face of the capsule so as to pierce said forward face and may then be engageable with the capsule to compress the capsule so as to dispense the material from the capsule to the discharge opening.

The invention will now be described by way of example and with reference to the accompanying schematic drawings wherein:

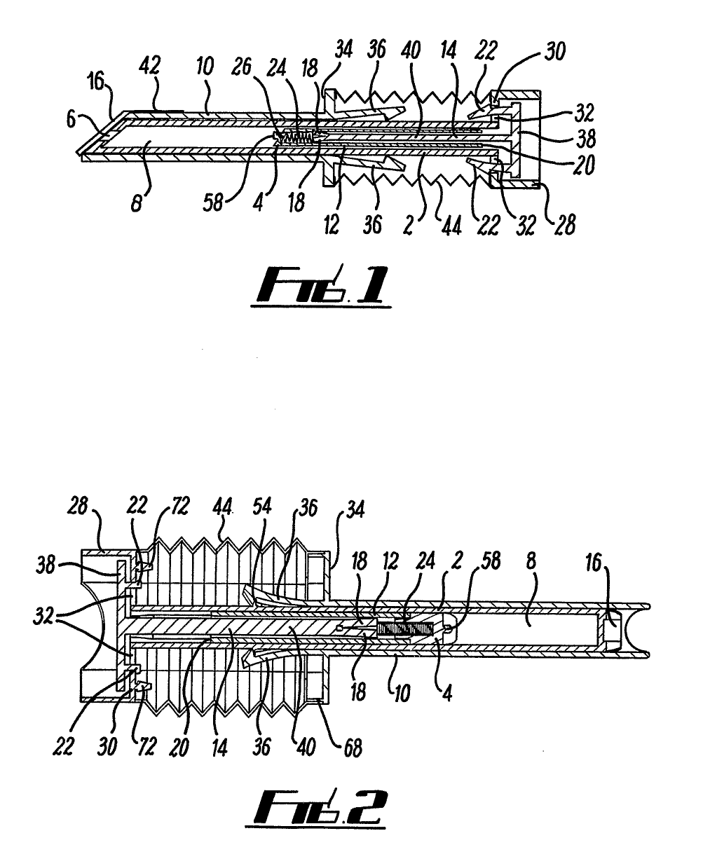

FIG. 1 is a longitudinal sectional view of an embodiment of a dosage delivery device, in a storage position, prior to use according to the present invention;

FIG. 2 is a longitudinal sectional view of a further embodiment of a dosage delivery device, in a storage position, prior to use according to the present invention;

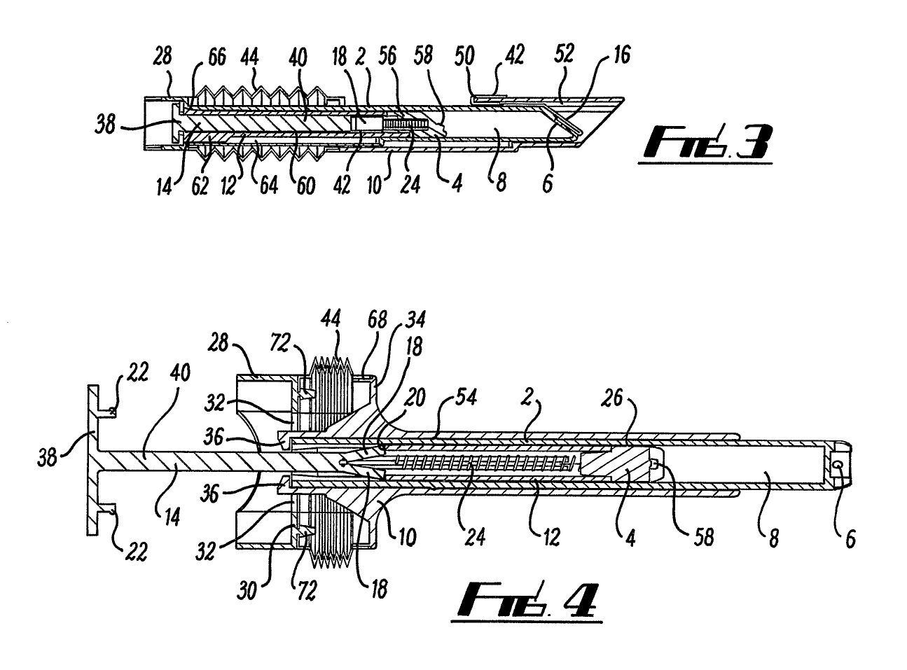

FIG. 3 is a longitudinal sectional view of the embodiment of FIG. 2, in a storage position, prior to use according to the present invention, in which the cross-section is taken at 90° to the cross-section in FIG. 2;

FIG. 4 is a longitudinal sectional view of the embodiment of FIG. 2, in a primed position;

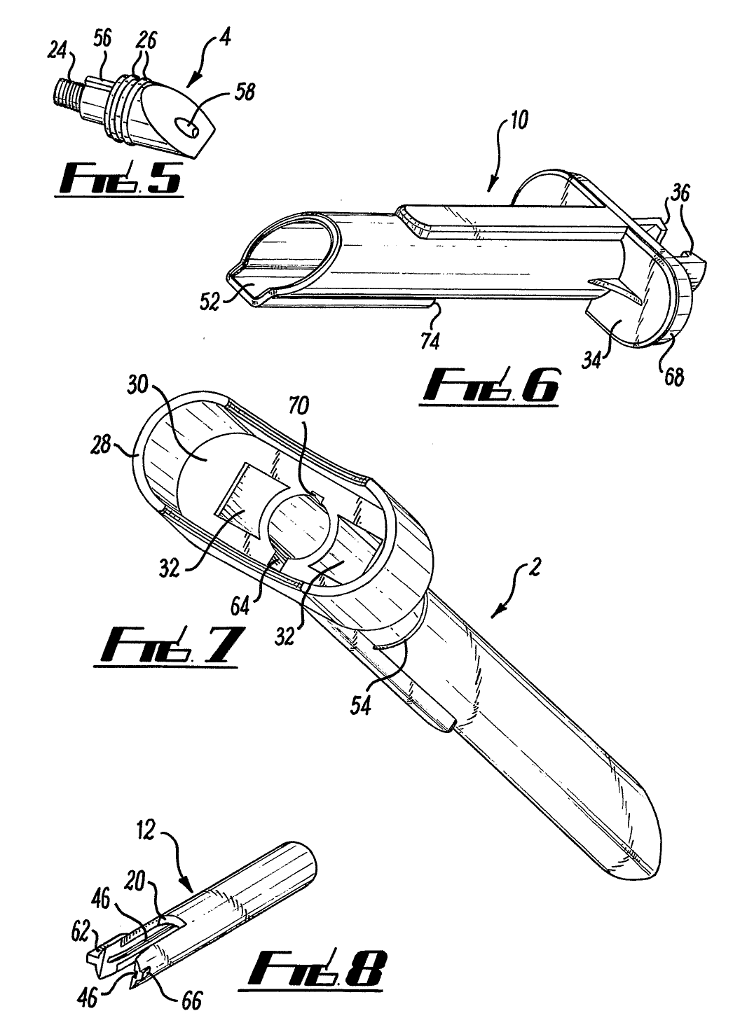

FIG. 5 is a front perspective view of the plunger of the embodiment of FIG. 2;

FIG. 6 is a front perspective view of the operating sleeve of the embodiment of FIG. 2;

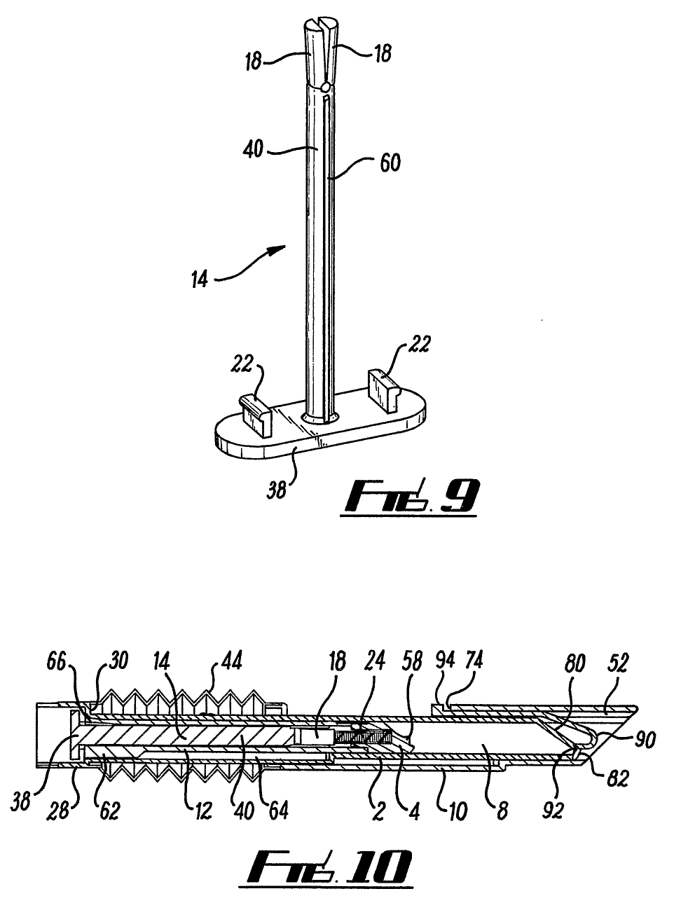

FIG. 7 is a rear perspective view of the housing of the embodiment of FIG. 2;

FIG. 8 is a rear perspective view of the injector sleeve of the embodiment of FIG. 2;

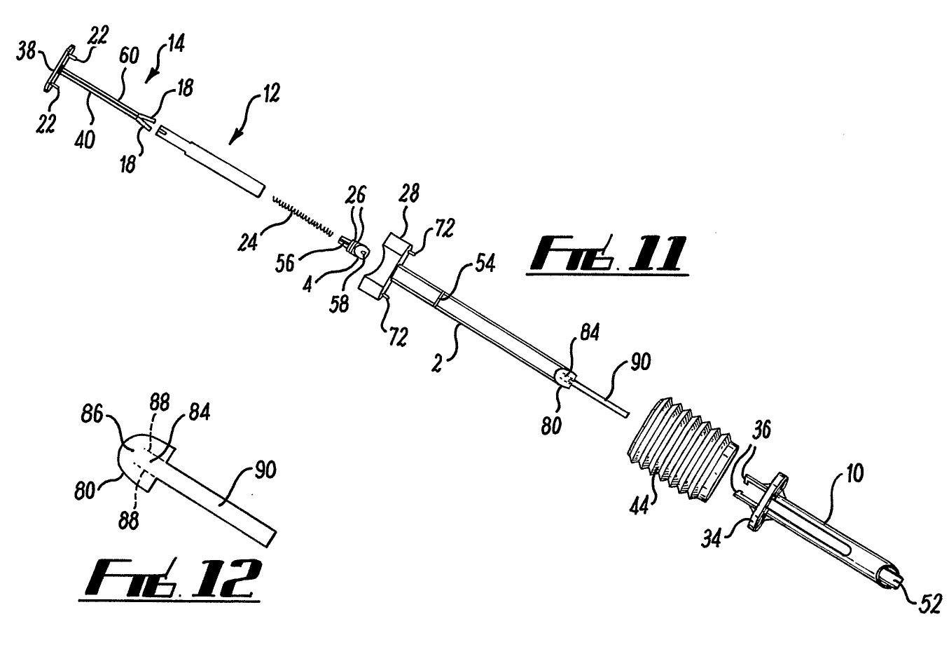

FIG. 9 is a front perspective view of the plunger actuator of the embodiment of FIG. 2;

FIG. 10 is a longitudinal sectional view of a further embodiment of a dosage delivery device, in a storage position, prior to use according to the present invention;

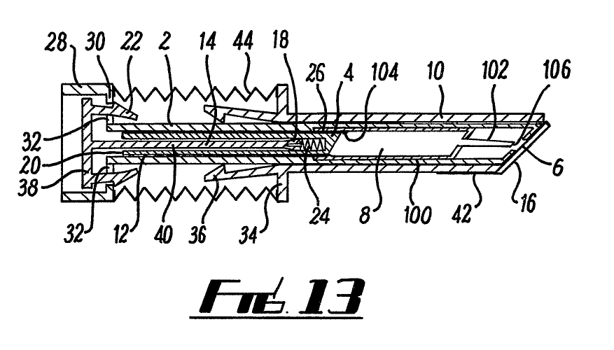

FIG. 11 is an exploded side perspective view of the components of the embodiment of FIG. 10;

FIG. 12 is a view of the forward face of the housing of the embodiment of FIG. 10, and

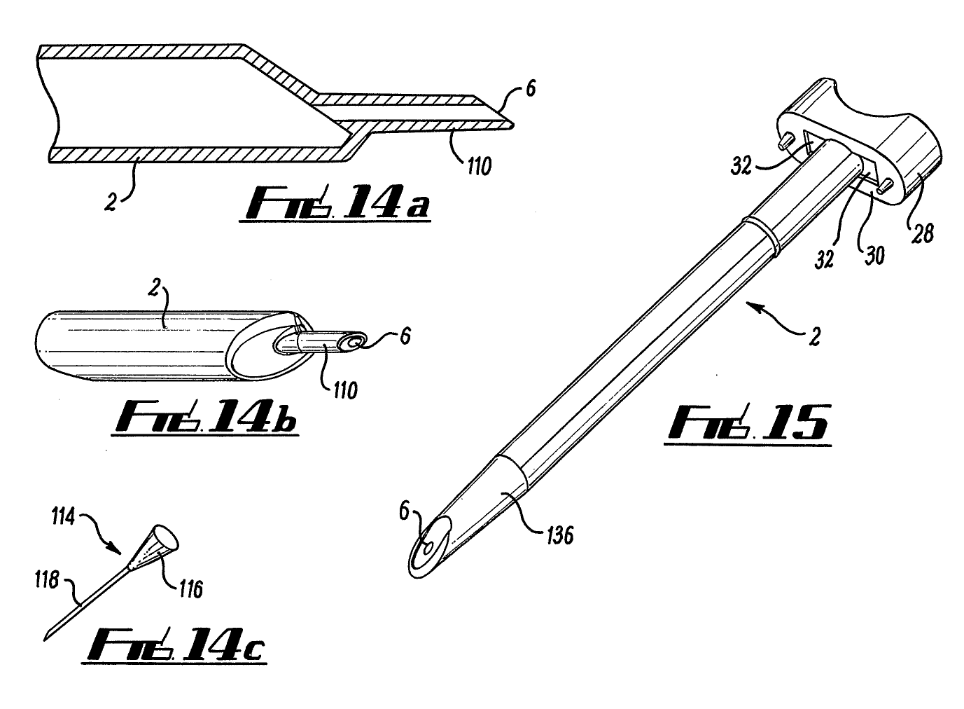

FIG. 13 is a longitudinal sectional view of a further embodiment of the present invention;

FIG. 14a is a longitudinal cross-section through and FIG. 14b is a front perspective view of a forward end of a housing of a dosage delivery device according to the present invention adapted to receive a luer slip needle attachment for delivery of a medicament by injection and FIG. 14c shows a luer slip needle attachment;

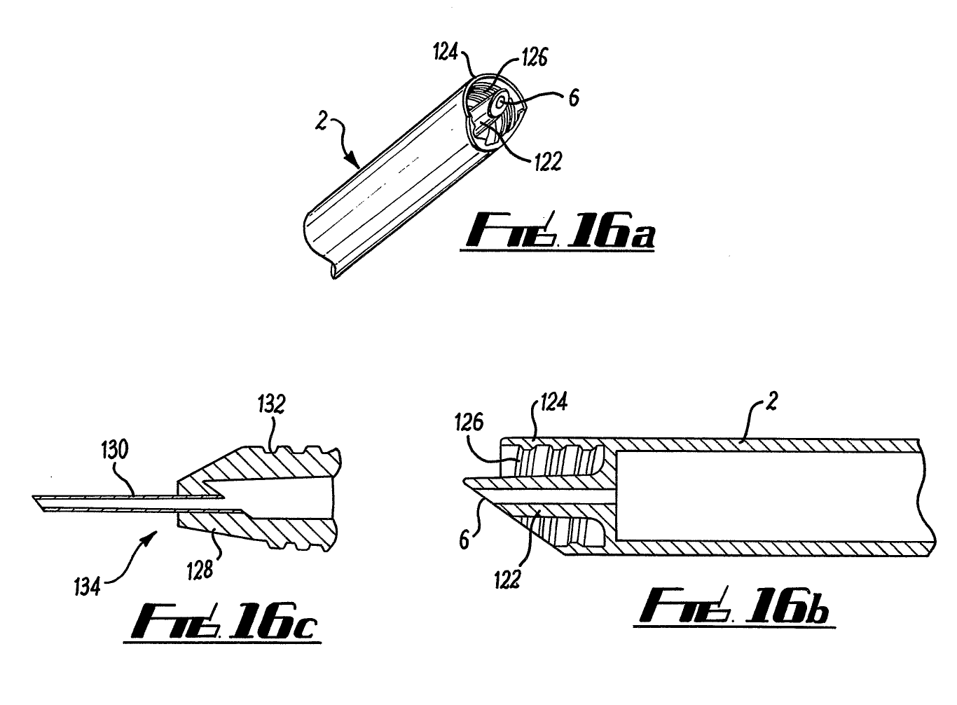

FIG. 15 is a front perspective view of a housing of a dosage delivery device according to the present invention adapted for rectal delivery of a medicament;

FIG. 16a is a front perspective view of a dosage delivery device and FIG. 16b a longitudinal cross-section through a forward end of a housing of a delivery device according to the present invention adapted to receive a luer lock needle attachment for delivery of a medicament by injection and FIG. 16c is a longitudinal cross-section through a luer lock needle attachment;

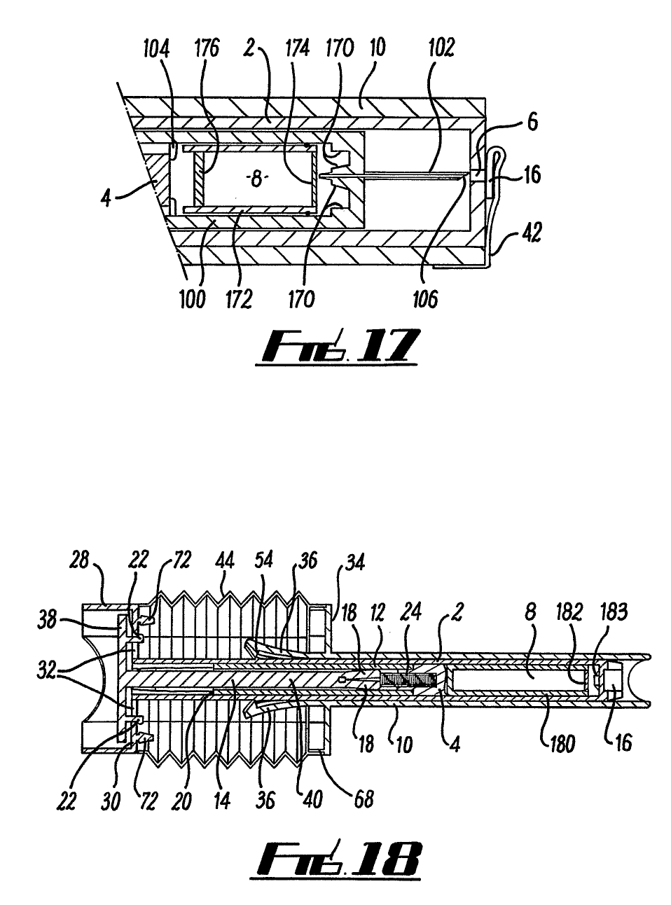

FIG. 17 is a longitudinal cross section through the front end of a further embodiment of the dosage delivery device, similar to that of FIG. 13, except with the medicament contained within a capsule; and

FIG. 18 is a longitudinal cross section of a further embodiment of the dosage delivery device, similar to that of FIG. 2, except with the medicament contained within a capsule.

The device shown in FIG. 1 comprises a generally cylindrical housing (2) defining a reservoir for holding a medicament (8). A plunger (4) is fitted within the reservoir with a ring seal (26) closing the rearward (right hand side) boundary of the reservoir as shown. At the forward end of the reservoir remote from the plunger (4) is formed a discharge opening (6) sealed by an adhesive strip (16). A tie (42) extends from the adhesive strip and is fixed to an outer surface of an operating sleeve (10). The rearward end of the housing (2) remote from the discharge opening is formed with an enlarged cylindrical portion (28). The enlarged portion (28) of the housing transitions into the remainder of the housing (2) via a step (30) within which are formed a pair of opposing latch holes (32).

Also, a spigot (58) extends from the forward face of the plunger (4) in a position corresponding to that of the discharge opening (6), so that when the forward face of the plunger engages the forward end of the housing (2), the spigot (58) moves into the discharge opening (6) so as to discharge any medicament located within the discharge opening.

The dosage delivery device comprises a plunger actuation mechanism which is moveable between a contracted storage position in which the mechanism is held at least partially within the housing (2) and an extended primed position in which the plunger (4) can be actuated. The plunger actuation mechanism comprises a generally cylindrical injector sleeve (12) mounted co-axially within the housing (2) and a plunger actuator (14) mounted co-axially within the injector sleeve (12) and thus within the housing (2). The dosage delivery device further comprises a priming mechanism slideably mounted on the housing (2) and moveable between a storage (FIG. 1) and a primed position to expose the discharge opening (6) and to release the plunger actuation mechanism (12, 14). The priming mechanism comprises a generally cylindrical operating sleeve (10) mounted co-axially around the housing (2).

The operating sleeve (10) is slideably mounted coaxially over the forward end of the housing, with the forward end of the operating sleeve aligned with the forward end of the housing, when the dosage delivery device is in a storage position, prior to use, as shown in FIG. 1. Towards the rearward end of the operating sleeve (10) is formed an outwardly extending flange (34), behind which the operating sleeve transitions into a pair of resilient opposing inwardly facing latches (36). Each latch (36) comprises a strip which extends rearwardly of the flange (34) and which is terminated by an inwardly facing triangular projection. In the storage position in FIG. 1, the inwardly facing latches (36) are urged radially outwardly against an inwardly directed biasing force due to the resilience of the latches, by the engagement of the triangular projections against the outer surface of the housing (2). In order that the latches (36) have the required resilience, the operating sleeve (10) may be made from a suitably resilient material, such as a mouldable or otherwise formable thermoplastic.

The injector sleeve (12) extends rearwardly of the plunger (4) and can be formed integrally with the plunger or can be a separate part fitted to or bearing against the plunger.

The plunger actuator (14) comprises a back plate (38) which is shaped to fit within the enlarged rearward portion (28) of the housing (2) and from which extends forwardly a central spur (40). The central spur extends co-axially within the injector sleeve (12) and is terminated at its end remote from the back plate (38) by a pair of opposing latch elements (18). At the opposite end of the central spur (40) from the plunger (4), are formed a pair of arms (18) which are normally biased resiliently outwardly relative to the axis of the central spur (40), but are constrained from so doing by the inner surface of the injector sleeve (12).

The plunger actuator (14) is telescopically received within the injector sleeve (12). A spring element (24), which is compressed in the storage position of the dosage delivery device shown in FIG. 1, is received within the injector sleeve (12) located between the rear of the plunger (4) and the arms (18). The spring element (24), which in FIG. 1 is shown as a helical spring, acts to bias the plunger actuator (14) rearwardly into a primed position.

A pair of outwardly facing latches (22) extend forwardly of the back plate (38) of the plunger actuator (14), one to either side of the central spur (40). The latches (22) each include a strip extending from the back plate, which is terminated at its end remote from the back plate by a triangular outwardly extending projection. The projections of the outwardly facing latches (22) engage the outer edge of the latch holes (32) formed in the step (30) of the housing (2) so as to hold the plunger actuator (14) within the housing (2) and injector sleeve (12) against the biasing force of the spring element (24). The engagement of the outwardly facing latches (22) of the plunger actuator (14) with the latch holes (32) of the housing (2) hold the dosage delivery device in its storage position, prior to use, as is shown in FIG. 1.

In order that the arms (18) and the latches (22) have the required resilience, the plunger actuator (14) may be made from a suitably resilient material, such as a mouldable or otherwise formable thermoplastic.

A compressible protective sleeve (44) is fitted between the rearward facing surface of the flange (34) of the operating sleeve (10) and the forward facing surface of the step (30) of the housing (2). The sleeve (44) is formed of as a concertina sleeve so as to facilitate compression of the sleeve and acts to conceal the pairs of latches (22, 36).

When the medicament (8) in the reservoir is required, the plunger actuator (14) is withdrawn from the injector sleeve (12) by operation of the operating sleeve (10). A user may locate their thumb over the rearward edge of the enlarged portion (28) of the housing and two fingers over the face of the flange (34) facing forwardly, away from the enlarged portion (28). Then a user may pull the flange (34) towards the step (30) of the housing (2). This draws the operating sleeve (10) rearwardly over the housing (2), pulling off the foil closure (16) to expose the discharge opening (6) and causing the compressible sleeve (44) to be compressed. The user pulls the flange (34) further towards the step (30) until the triangular projections of the outwardly and inwardly facing latches (22, 36) engage so that the inwardly facing latches (36) push the outwardly facing latches (22) inwardly (towards the longitudinal axis of the housing (2)) so as to disengage the outwardly facing latches (22) from the latch holes (32) in the step (30) of the housing (2). The user then continues to pulls the flange (34) further towards the step (30) until the inwardly facing latches (36) engage the inner edges of the latch holes (32) so as to hold the operating sleeve (10) in a rearward primed position on the housing (2).

As soon as the outwardly facing latches (22) of the plunger actuator (14) disengage from the latch holes of the housing (2), the spring element (24) urges the plunger actuator (14) rearwardly until the arms (18) of the plunger actuator leave the rearward end of the injector sleeve (12). The arms (18) move away from each other to abut the inner surface of the housing (2) to the rear of the injector sleeve (12) and are engageable with the rearward end (20) of the injector sleeve (12) when the plunger actuator (14) is depressed (moved forward).

The plunger actuator (14) can then be depressed as if it were part of a conventional syringe, and by virtue of the arms (18) engaging the rearward face (20) of the injector sleeve (12), depression of the plunger actuator (14) causes the injector sleeve (12) to move the plunger (4) forwardly towards the discharge opening (6). In this way, liquid medicament (8) is discharged from the reservoir, through the discharge opening (6), until the forward face of the plunger (4) abuts the end face of the housing (2).

The housing (2) in the device of FIG. 1 defines a generally cylindrical reservoir which has an end face in which is located the discharge opening (6), which end face is angled with respect to the longitudinal axis of the housing (2). The plunger (4) on its side facing the discharge opening (6), is formed with a correspondingly angled end face. With these matching profiles, when the plunger reaches its forward most position, the forward face of the plunger (4) and the end face of the housing (2) are complementarily engaged, with only a minimal volume (of medicament) remaining in the reservoir upstream of the opening (6).

In FIGS. 2 to 9 a generally similar device is illustrated, with like parts denoted by like numerals.

The device shown in FIGS. 2 to 9 comprises the generally cylindrical housing (2) defining the reservoir for holding the medicament (8).

As shown in FIG. 7, the rearward end of the housing (2) remote from the discharge opening is formed with the enlarged generally oval shaped sleeve portion (28). The enlarged portion (28) of the housing transitions into the remainder of the housing (2) via the step (30) within which are formed the pair of opposing latch holes (32). In this embodiment, the housing (2) is formed on its outer surface with a pair of opposing ledges (54) which engage the inwardly facing latches (36) in the stored position, as shown in FIG. 2, so as to limit forward movement of the operating sleeve (10) on the housing (2).

The dosage delivery device comprises a plunger actuation mechanism which is moveable between a contracted storage position in which the mechanism is held at least partially within the housing (2) and an extended primed position in which the plunger (4) can be actuated. The plunger actuation mechanism comprises a generally cylindrical injector sleeve (12) mounted co-axially within the housing (2) and a plunger actuator (14) mounted co-axially within the injector sleeve (12) and thus within the housing (2). The dosage delivery device further comprises a priming mechanism slideably mounted on the housing (2) and moveable between a storage (FIG. 1) and a primed position to expose the discharge opening (6) and to release the plunger actuation mechanism (12, 14). The priming mechanism comprises a generally cylindrical operating sleeve (10) mounted co-axially around the housing (2),

The flange (34) formed on the operating sleeve (10) is generally oval shaped so as to match the shape of the enlarged portion (28) of the housing (2). The protective sleeve (44) is correspondingly shaped and the flange (34) is formed with a rearwardly extending rim (68) on which the forward end of the protective sleeve (44) is mounted. The step (30) of the housing (2) is formed on its forward facing side with a pair of locking projections (72) over which an inwardly projecting rim of the rearward end of the protective sleeve (44) can be secured. A pair of holes is formed in the inwardly projecting rim of the protective sleeve (44) through which the locking projections are pushed so as to secure the sleeve (44) to the housing (2).

A rearward end of the plunger (4) is press fit within a forward end of the injector sleeve (12), with a key or guiding ridge (56) (shown in FIG. 5) being formed on the periphery of the plunger which fits within a corresponding keyway formed in the injector sleeve so as to hold the plunger (4) and injector sleeve (12) against relative rotation. As shown in FIG. 5, a pair of ring seals (26) is formed integrally with the plunger for providing a seal between the plunger and the rearward end of the reservoir. Also, a spigot (58) extends from the forward face of the plunger (4) in a position corresponding to that of the discharge opening (6), so that when the forward face of the plunger engages the forward end of the housing (2), the spigot (58) moves into the discharge opening (6) so as to discharge any medicament located within the discharge opening.

The plunger actuator (14) is shown in more detail in FIG. 9, which shows one of a pair of guiding ridges or keys (60) which extends along the length of opposing sides of the central spur (40) and engage a pair of corresponding keyways (46) formed in opposite sides of the inner surface of the injector sleeve (12) so as to prevent relative rotation between the injector sleeve (12) and the plunger actuator (14). Furthermore, the outwardly facing latches (22) of the plunger actuator are formed with shorter stems, and rounded outward projections which are smaller than, those described above in relation to FIG. 1.

As shown in FIG. 8, the injector sleeve is formed with a pair of opposing slots at its rearward end. The forward end faces (20) of these slots provide the rearward faces of the injector sleeve (12) against which the arms (18) of the plunger actuator (14) bear when the dosage delivery device is primed. The slots formed in the injector sleeve are wide enough to receive the arms (18) of the plunger actuator.

To one side of the pair of slots, the injector sleeve (12) is formed with a key or guiding ridge (62) in its outer surface with fits slideably along a corresponding keyway (64) formed in the housing (2). At the opposite side of the injector sleeve (12) to the key (62) is formed a sloping braking ridge (66) which fits into a correspondingly shaped slot (70) formed in the housing (2). The engagement of the braking ridge (66) with the slot (70) secures the injector sleeve (12) within the housing (2) against forward movement until the dosage delivery device is primed.

As is shown in FIG. 3, an adhesive foil closure (16) is fitted over the discharge opening (6). The side of the foil closure (16) adjacent to the slot (52) in the operating sleeve (10) has a first end of a tie (42) fitted to it. The tie (42) is threaded rearwardly along the slot (52) and passed around a rearward end of the slot (52) and then fixed to the outer surface of the operating sleeve (10).

In the storage position of the device shown in FIGS. 2 and 3, the forward end of the operating sleeve (10) extends slightly forwardly of the forward end of the housing (2) so as to provide protection for the foil covered discharge opening (6). The operating sleeve (10) is secured against forward movement on the housing (2) by the engagement of the inwardly facing latches (36) of the operating sleeve, engaging the ridges (54) formed on the housing (2). The plunger (4) is secured against forward movement within the reservoir by the engagement of the braking ridge (66) of the injector sleeve (12) with the housing (2). The plunger actuator (14) is held against the biasing force of the spring element (24) within the injector sleeve (12) by the engagement of the outwardly facing latches (22) with the latch holes (32) in the step (30) of the housing (2).

When the medicament (8) in the reservoir is required, a user pulls the flange (34) of the operating sleeve (10) towards the step (30) of the housing (2). This draws the operating sleeve (10) rearwardly over the housing (2), pulling off the foil closure (16) to expose the discharge opening (6) and causing the compressible sleeve (44) to be compressed. As the operating sleeve moves rearwardly with respect to the housing (2), the tie (42) draws the foil closure (16) from the discharge opening and into the slot (52) formed at the forward end of the operating sleeve. The foil closure (16) is then safely stored within the slot (52). The user continues to pull the flange (34) towards the step (30) so that the triangular projections of the inwardly facing latches (36) engage the rounded projections of the outwardly facing latches (22) so that the inwardly facing latches (36) push the outwardly facing latches (22) inwardly (towards the longitudinal axis of the housing (2)) so as to disengage the outwardly facing latches (22) from the latch holes (32) in the step (30) of the housing (2). The user continues to pull the flange (34) further towards the step until the inwardly facing latches (36) engage the inner edges of the latch holes (32) so as to hold the operating sleeve (10) in a rearward primed position on the housing (2), as shown in FIG. 4.

As soon as the outwardly facing latches (22) of the plunger actuator (14) disengage from the latch holes of the housing (2), the spring element (24) urges the plunger actuator (14) rearwardly until the arms (18) of the plunger actuator approach the rearward end of the injector sleeve (12). The arms (18) move away from each other, into the pair of opposing slots at the rear of the injector sleeve (12) to abut the inner surface of the housing (2) to the rear of the injector sleeve (12). The arms are then engageable with the rearward faces (20) of the injector sleeve (12) when the plunger actuator (14) is depressed (moved forward) (See FIG. 4).

The plunger actuator (14) can then be depressed as if it were part of a conventional syringe, and by virtue of the arms (18) engaging the surfaces (20) of the injector sleeve (12), depression of the plunger actuator (14) causes the injector sleeve (12) to move the plunger (4) forwardly towards the discharge opening (6). In this way, liquid medicament (8) is discharged from the reservoir, through the discharge opening (6), until the forward face of the plunger (4) abuts the end face of the housing (2).

A further embodiment of the present invention is shown in FIGS. 10 to 12 with like parts to the previous embodiments shown with like numerals. The embodiment of FIGS. 10 to 12 is very similar to that of FIGS. 2 to 9, except that the foil closure (16) and tie (42) are replaced by a closure arrangement (84, 90) which is integrally formed with the housing (2).

In the embodiments of FIGS. 10 to 12, the front end of the housing (2) is formed as a sloping end face (80) which tapers to a nose portion (82) of the end face. The sloping end face (80) tapers so that the nose portion (82) is remote from the slot (52) in the operating sleeve. The end face (80) is formed with a closure (84) defined by a hinge region (86) at the end of the closure remote from the nose portion (82) and a pair of frangible tear lines (88) extending from the hinge region to the nose portion (82). The end of the closure (84) remote from the hinge region (86) meets the nose portion (82) at a third frangible tear line (92) and then extends therefrom to form a tie (90) which extends as a strip from the forward end of the housing (2). The closure (84) and the tie (90) are formed integrally with the housing, for example in an injection moulding process. The end of the tie (90) remote from the housing (2) is formed with a projection (94) (FIG. 10).

In order to mount the housing (2) within the operating sleeve (10) the tie (90) is folded back, as is shown in FIG. 10 and then the operating sleeve (10) is slideably mounted over the front of the housing (2) and moved rearwardly. The slot (52) formed in the operating sleeve (10) is aligned with the tie (90) so that the folded back tie (90) is held within the slot. The projection (94) at the end of the tie engages the rearward facing edge of the slot (52) in the operating sleeve (10).

Then in order to open the discharge opening of the housing (2) the closure (84) must be opened. This is done as part of the priming of the dosage delivery device, in a similar way as is described above in relation to FIGS. 2 to 9.

In order to dispense a medicament, the flange (34) of the operating sleeve (10) is drawn rearwardly towards the enlarged portion (28) of the housing. During this movement, the rearward edge (74) of the slot (52) of the operating sleeve, engages the projection (94) on the tie (90) to pull the tie (90) rearwardly. This causes the frangible tear lines (92, 88) to tear and then pull the closure (84) rearwardly into the slot (52) so that the discharge opening is opened. Priming then continues to release the plunger actuator (14) in the same way as is described above in relation to FIGS. 2 to 9.

It will be recognised that as a pre-charged dosage delivery unit, the device of the present invention can be activated single-handedly and used extremely swiftly. Indeed, the priming of the device is affected by a single movement of the operating sleeve (10). This is of self-evident benefit when the device has to be used in emergency situations. The device is of benefit in the field of human and animal medicine.

The embodiments described above have a housing (2) with a forward end generally shaped to deliver a medicament orally. The housing (2) can also be adapted to deliver a medicament rectally, as is described below in relation to FIG. 15, with like parts indicated by like numerals.

In addition, the housing (2) can be adapted to deliver a liquid medicament via a needle, as is known in the art. The adaptation of the devices described above can simply be achieved by making the forward end of the housing (2), suitable for receiving an injection needle luer attachment, as is described below in relation to FIGS. 14 and 16, with like parts identified by like numerals. Alternatively, the housing (2) can be adapted as shown in FIG. 13, again with like parts represented by like numerals.

In the arrangement of FIG. 13, the housing (2) is formed with an increased internal diameter portion towards its forward end, within which is slideably located a secondary reservoir (100). The secondary reservoir has a rearward end which fits around the forward end of the plunger (4) and a forward end within which is fitted an injection needle (102). The injection needle has a discharge opening (106) located at its forward end and which is aligned with the opening (6) of the housing (2). A space is provided between a forward end of the secondary reservoir (100) and the forward face of the housing (2) within which the needle (102) can be housed within the housing (2). An internal braking ridge (104) is formed on the internal surface of the secondary reservoir (102) adjacent the forward end of the plunger (4) in the storage position, shown in FIG. 13.

When a medicament (8) is to be dispensed from the device of FIG. 13, the operating sleeve (10) is used to release the plunger actuating mechanism (12, 14) and to expose the discharge opening of the needle (106) by removing the closure member (16), as is described above in relation to FIG. 1. Then when a user depresses the plunger (4), with a first predetermined force, the plunger first pushes the secondary reservoir forwards, by the engagement of the plunger (4) with the braking ridge (104) of the secondary reservoir. As the user continues to depress the plunger (4), the forward face of the reservoir (100) moves into abutment with the forward face of the housing (2) and the needle (102) extends from the opening (6) in the front of the housing (2). The user can recognise this abutment, as an increased force is then required to depress the plunger (4) further. Then with further depression of the plunger (4), with a force greater then the first predetermined force, the plunger overrides the braking ridge (104) to move forwards along the secondary reservoir (100) so as to dispense medicament from the discharge opening (106) of the needle (102).

FIG. 15 shows a housing (2) with a forward end (136) adapted to deliver a dose of medicament rectally. The forward end of the housing (2) is formed with a long tapered nozzle (136), narrowing from the remainder of the housing (2) and suitable for placement within the rectum of a patient and the discharge opening (6) is located at the forward end of the nozzle (136). In use, the dosage delivery device is primed by pulling the operating sleeve (10) rearwardly with respect to the housing (2) so as to expose the discharge opening (6) and to release the plunger actuator (14). Then the nozzle (136) is placed into a patient's rectum and the medicament (8) administered by depressing the plunger actuator (14).

FIGS. 14a and 14b show an alternative design of housing (2) having a forward end formed with a leur slip nozzle (110) adapted to receive a standard luer slip needle attachment (114), an example of which is shown in FIG. 14c. The luer slip nozzle has a reduced diameter as compared to the remainder of the housing (2). The luer slip needle attachment comprises a cup (116) for fitment over the nozzle (110) and a needle (118) embedded within the cup at an apex of the cup (116). Such luer slip needle attachments are well known in the art. The discharge opening (6) is formed at the forward end of the luer slip nozzle (110)

In use, the dosage delivery device is primed by pulling the operating sleeve (10) rearwardly with respect to the housing (2) so as to expose the discharge opening (6) and to release the plunger actuator (14). Then the luer slip needle attachment (114) is fitted over the luer slip nozzle (110) so as to cover the discharge opening. This is done by sliding the cup portion (116) of the attachment (114) over the luer slip nozzle (110) so that the needle (118) is in communication with the discharge opening (6). Then the needle (118) is vented of air by depressing the plunger actuator. The needle (118) is then inserted into a patient and the medicament (8) is administered to the patient via the needle (118) by further depressing the plunger actuator (14).

FIGS. 16a and 16b show an alternative design of housing (2) having a forward end adapted to receive a standard luer lock needle attachment, which are well known in the art and an example of which is shown in FIG. 16c. The forward end of the housing (2) is formed with a nozzle (122) which is narrower than the remainder of the housing. The discharge opening (6) is formed at the forward end of the nozzle (122). The nozzle (122) is surrounded by a cylindrical sleeve (124) which is a forward extension of the cylindrical sidewall of the housing (2). The nozzle (122) and cylindrical sleeve (124) are co-axial and the forward end of the nozzle (122) projects from the forward end of the cylindrical sleeve (124). The cylindrical sleeve (124) is formed with a helical thread (126) in its internal surface, which faces towards the nozzle. The luer lock needle attachment (134) is formed of a cup shaped main body (128) with an injection needle (130) embedded in and extending from an apex of the main body at the forward end of the main body (128). The cup shaped main body (128) has a central cavity shaped to match the profile of the nozzle (122) of the housing (2) over which it is adapted to fit. The main body (128) is also formed with an external screw thread (132) which fits the internal screw thread (126) of the cylindrical sleeve (124) at the front end of the housing (2). Thus the cup shaped body (128) can be screw threadedly mounted between the nozzle (122) and cylindrical sleeve (124) of the housing (2) so that the needle (130) of the luer lock needle attachment (134) is fitted over the discharge opening (6) of the housing (2).

In use, the dosage delivery device is primed by pulling the operating sleeve (10) rearwardly with respect to the housing (2) so as to expose the discharge opening (6) and to release the plunger actuator (14). Then the luer lock needle attachment (134) is screw threadedly fitted over the nozzle (122) of the housing (2) so as to cover the discharge opening (6). This is done by placing the cup shaped main body (128) of the attachment over nozzle (122) at the forward face of the housing (2) and twisting the main body (128) with respect to the housing (2) so as to screw threadedly mount the attachment (134) to the housing via the engagement of the screw thread (132) on the main body (128) with the internally threaded surface of the sleeve (124) of the housing (2). Then the needle (130) is vented of air by depressing the plunger actuating mechanism and then inserted into a patient and the medicament (8) is administered to the patient via the needle by further depressing the plunger actuator (14).

Therefore, the present invention could also have benefit in vaccination programmes, where a single injection can be completed within a very short time. This also means that an individual being vaccinated can have minimal advance sight of the device, or more particularly a vaccination needle. It could also be of use for the delivery of a pre-measured dose of medicament, such as morphine, in an emergency situation, such as a hospital or war zone, for emergency pain relief. It may also be of use where regular pre-measured dosages of medicament are required to be self-injected by a patient, such as in the case of diabetes.

FIG. 17 shows an embodiment similar to that of FIG. 13, with like parts labelled with like numerals, except that the medicament (8) is contained within a sealed cylindrical capsule (172). The secondary reservoir (100) is slideably located within the increased diameter portion of the housing (2). The forward portion of the plunger (4) is located within a rearward portion of the secondary reservoir (100), with an annular gap between the external surface of the forward portion of the plunger and the internal surface of the rearward portion of the secondary reservoir. The secondary reservoir (100) has a forward end face through which is fitted an injection needle (102). The injection needle has a discharge opening located at its forward end which is aligned with the opening (6) of the housing (2). The rearward end of the needle (102) extends into the secondary reservoir (100) and a set of stops (170) are formed behind the forward end face of the secondary reservoir. A cylindrical capsule (172), filled with a medicament (8) is slideably located within the secondary chamber (100). The capsule (172) has a forward end face (174) formed from a pierceable membrane. The rearward end of the capsule (172) is closed off by a capsule plunger (176), which can be moved forwardly within the capsule (172) (towards the membrane (174)). A pair of opposing internal braking ridges (104) are formed on the internal surface of the secondary reservoir (100), adjacent the forward end of the plunger (4) in the storage position. The forward end of the plunger (4) is shaped to slideably fit within the cylindrical capsule (172).

When a medicament (8) is to be dispensed from the device of FIG. 17, the operating sleeve (10) is used to release the plunger actuating mechanism (12, 14) and to expose the opening (6) in the housing (2) so that the discharge opening of the needle (106) is exposed, by removing the closure member (16), as is described above in relation to FIG. 1. Then when a user depresses the plunger (4), with a first predetermined force, the plunger first pushes the secondary reservoir (100) forwards, by the engagement of the plunger (4) with the braking ridges (104) of the secondary reservoir. As the user continues to depress the plunger (4), the forward face of the reservoir (100) moves into abutment with the forward face of the housing (2) and the needle (102) extends from the opening (6) in the front of the housing (2). The user can recognise this abutment, as an increased force, and then required depresses the plunger (4) further. Then with the further depression of the plunger (4), with a force greater then the first predetermined force, the plunger overrides the braking ridge (104) to move forwards into the rearward end of the capsule (172). The plunger (4) engages the capsule plunger (176), however, as the capsule (170) is closed, the forward movement of the plunger (4) acts to move the capsule (172) forward slideably within the secondary reservoir (100) until the capsule membrane (174) is pushed onto the rearward end of the needle (102) and is pierced by the needle and the forward end of the capsule engages the stops (170). The rearward end of the needle (102) is then in communication with the medicament (8). Thereafter, because the capsule is now vented further forward movement of the plunger (4) moves the capsule plunger towards the membrane (174) so as to dispense the medicament (8) from the discharge opening (106) of the needle (102).

The arrangement shown in FIG. 18 is similar to that described in FIG. 2, except that the medicament (8) is contained within a sealed cylindrical capsule (180). The capsule (180) is located within the housing (2) with its rearward end adjacent the forward end of the plunger (4) in the storage position. The forward end of the capsule (180) is formed from a pierceable membrane (182) and the remainder of the capsule is formed from a deformable material. The capsule (180) is positioned so that there is a small gap between the membrane (182) and the forward face of the housing (2). A U-shaped ridge (183) is formed around the discharge opening (6), which ridge extends towards the membrane (182). The peak of the U-shaped ridge (183) is sharp.

When a medicament (8) is to be dispensed from the device of FIG. 18, the operating sleeve (10) is used to release the plunger actuating mechanism (12, 14) and to expose the discharge opening of the housing (2) by removing the closure member (16), as is described above in relation to FIG. 2. Then when a user depresses the plunger (4), the plunger pushes the capsule (180) forwardly within the housing, until the membrane (182) is pushed onto the U-shaped ridge (183) and the U-shaped ridge makes a U-shaped cut in the membrane (182), via which medicament (8) can be dispensed from the capsule (180) into the discharge opening (6). Further forward movement of the plunger (4) compresses the deformable capsule (180), because the capsule is now vented, so as to dispense medicament from the discharge opening (6) of the housing (2).

1-32. (canceled)

33. A dosage delivery apparatus, comprising:
a housing;
a reservoir located within said housing;
a plunger located within said reservoir and movable for dispensing a material from a discharge opening of said reservoir;
a plunger actuation mechanism having a contracted storage position wherein said plunger actuation mechanism is, at least partially, held within said housing and having an extended primed position wherein said plunger is able to be actuated and wherein said plunger actuation mechanism is movable between the contracted storage position and the extended primed position from which said plunger actuation is able to be actuated; and,
a priming mechanism slidably mounted relative to said housing and having a storage position wherein said plunger actuation mechanism is in the contracted storage position and a primed position, wherein said plunger actuation mechanism is in the extended primed position and wherein said priming mechanism is movable between the storage position and the primed position for exposing the discharge opening of said reservoir and for releasing said plunger actuation mechanism into the extended primed position.

34. The dosage delivery apparatus according to claim 33, wherein said plunger actuation mechanism is biased into the extended primed position.

35. The dosage delivery apparatus according to claim 33, further comprising a latch arrangement wherein said plunger actuation mechanism is, at least partially, held within said housing via said latch arrangement, and said priming mechanism comprises an abutment surface for engaging said latch arrangement for releasing said plunger actuation mechanism.

36. The dosage delivery apparatus according to claim 35, wherein said latch arrangement is outwardly facing and engages a portion of said housing and said abutment surface comprises an inwardly facing latch arrangement.

37. The dosage delivery apparatus according to claim 35, wherein said housing includes a latch hole for engaging said latch arrangement on said plunger actuation mechanism in the storage position and for engaging said latch arrangement on said priming mechanism in the primed position.

38. The dosage delivery apparatus according to claim 37, wherein said latch arrangement on said plunger actuation mechanism engages the latch one from a first side and said latch arrangement on said priming mechanism engages the latch hole from a second side with the second side being opposite the first side.

39. The dosage delivery apparatus according to claim 37, wherein said housing is substantially cylindrical and transitions into an enlarged rearward portion via a stepped portion comprising an outwardly extending step face and latch holes being formed though said outwardly extending step face.

40. The dosage delivery apparatus according to claim 33, wherein said priming mechanism comprises a latch arrangement engageable with said housing in the primed position.

41. The dosage delivery apparatus according to claim 33, wherein said plunger actuation mechanism comprises an injection sleeve slidably mounted within said housing behind, and operatively engaged with, said plunger, said plunger actuation mechanism including a plunger actuator slidably mounted within said injection sleeve and engagable with said housing in the storage position and releasable from said housing and engagable with said injection sleeve in the primed position for actuating said plunger.

42. The dosage delivery apparatus according to claim 41, further comprising a spring arrangement located within said injection sleeve between said plunger and said plunger actuator for urging said plunger actuator to the primed position when said plunger actuator is released from said housing.

43. The dosage delivery apparatus according to claim 33, further comprising a closure member and a tie wherein, in the storage position, said closure member covers the discharge opening of said reservoir and said tie is fixed between the discharge opening and said priming mechanism for facilitating removal of said closure member for exposing the discharge opening when said priming mechanism is moved between the storage position and the primed position.

44. The dosage delivery apparatus according to claim 33, further comprising a closure member wherein said priming mechanism includes an operating sleeve slidably mounted on said housing having a forward portion operatively connected to said closure member of the discharge opening of said reservoir and having a rearward portion engagable with said plunger actuation mechanism for releasing said plunger actuation mechanism, wherein said priming mechanism moves rearwardly along said housing between the storage position and the primed position.

45. The dosage delivery apparatus according to claim 33, wherein said housing includes an enlarged portion at a rearward end of said housing and said priming mechanism includes an enlarged portion and a compressible protective sleeve mounted between said enlarged portion of said housing and said enlarged portion of said priming mechanism, wherein movement of said priming mechanism between the storage position and the primed position moves said enlarged portion of said housing and said enlarged portion of said priming mechanism together compressing said compressible protective sleeve.

46. The dosage delivery apparatus according to claim 33, further comprising:
a secondary reservoir containing the material slidably located within said housing; and,
an injection needle communicating with material within said secondary reservoir and extending forwardly of said secondary reservoir within said housing and aligned with a discharge opening of said housing,
wherein said plunger is, initially, engagable with said secondary reservoir for sliding said secondary reservoir forward within said housing and moving said injection needle through the discharge opening of said housing and, then, slidable within said secondary reservoir for dispensing the material through said injection needle.

47. The dosage delivery apparatus according to claim 46, wherein said reservoir contains a sealed capsule containing the material.

48. The dosage delivery apparatus according claim 47, wherein said sealed capsule has a pierceable forward face and said secondary reservoir comprises a cutting element for piercing the forward face of said sealing capsule for dispensing material from within said sealing capsule via the forward face of said sealing capsule following piercing of the forward face to the discharge opening of said housing.

49. The dosage delivery apparatus according to claim 48, wherein said sealed capsule is compressible via engagement with said plunger for dispensing the material.

50. The dosage delivery apparatus according to claim 49, wherein said plunger is engagable with said sealed capsule for sliding said sealed capsule forward within said secondary reservoir until the cutting element engages the pierceable forward face of said sealed capsule for piercing the forward face and then engaging with said sealed capsule for compressing said sealed capsule for dispensing the material from said sealed capsule to the discharge opening.

51. The dosage delivery apparatus according to claim 33, further comprising:
a secondary reservoir containing a sealing capsule, having a pierceable forward face, slidably located within said housing; and,
an injection needle having a rearward end forming a cutting element and a forward end extending forward of said secondary reservoir within said housing and aligned with the discharge opening of said housing,
wherein said plunger is, firstly, engagable within said secondary reservoir for sliding said secondary reservoir forward within said housing and for moving said injection needle through the discharge opening is, secondly, engagable with said sealing capsule for sliding said sealing capsule forward within said secondary reservoir until the rearward end of said injection needle engages the pierceable forward face of said sealing capsule for piercing the pierceable forward face and, thirdly, is engagable within said sealing capsule for dispensing the material through said injection needle.

52. A method for operating a dosage delivery apparatus having:
a housing;
a reservoir located within said housing;
a plunger located within said reservoir and movable for dispensing a material from a discharge opening of said reservoir;
a plunger actuation mechanism having a contracted storage position wherein said plunger actuation mechanism is, at least partially, held within said housing and having an extended primed position wherein said plunger is able to be actuated and wherein said plunger actuation mechanism is movable between the contracted storage position and the extended primed position from which said plunger actuation is able to be actuated; and,
a priming mechanism slidably mounted relative to said housing and having a storage position wherein said plunger actuation mechanism is in the contracted storage position and a primed position, wherein said plunger actuation mechanism is in the extended primed position and wherein said priming mechanism is movable between the storage position and the primed position for exposing the discharge opening of said reservoir and for releasing said plunger actuation mechanism into the extended primed position,
said method comprising the steps of:
moving said priming mechanism from the stored portion to the primed position; and,
actuating said plunger using said plunger actuating mechanism for dispensing the material from the discharge opening.
